(19) United States
(12) Patent Application Publication
(10) Pub. No.: US 2009/0171463 A1
(43) Pub. Date: Jul. 2, 2009

(54) Arthrodesis module and method for providing a patient with an arthrodesis

(76) Inventor: Peter Brehm, Weisendorf (DE)

Correspondence Address: PAUL A. FATTIBENE;FATTIBENE & FATTIBENE, 2480 POST ROAD, SOUTHPORT, CT, 06890, US

(21) Appl. No.: 12/317,141

(22) Filed: Dec. 19, 2008

Related U.S. Application Data
(60) Provisional application No. 61/009,410, filed on Dec. 28, 2007.

(51) Int. Cl. A61F 2/38; A61B 19/00

(52) U.S. Cl. 623/20.14; 623/20.15; 623/20.21; 623/20.24; 128/898

(57) ABSTRACT

An endoprothetical connection element for creating a knee arthrodesis comprises a coupling element that comprises a first fastener and a second fastener. The first fastener is adapted for connection with a first fastening element corresponding to the first fastener that is provided at a shaft insertable into a femur of a patient. The second fastener is adapted for connection with a second fastening element corresponding to the second fastener that is provided at a shaft insertable into a tibia of the patient. The coupling element is formed such that a first longitudinal axis of the femoral shaft connected to the first fastener and a second longitudinal axis of the tibial shaft connected to the second fastener include an angle different from 180 degrees which, when implanted into the body of the patient, is lying in a coronal plane of the patient.

### RELATED APPLICATION

This application claims the benefit of U.S. Provisional Application No. 61/009,410, filed Dec. 28, 2007.

### FIELD OF THE DISCLOSURE

The present disclosure relates to endoprostheses, in particular to endoprostheses for creation of a knee arthrodesis, as well as to a modular system wherein a knee arthrodesis and/or an artificial hip joint may be assembled according to requirements. The present disclosure further relates to a method for the creation of a knee arthrodesis and to a method for providing a patient with an endoprosthesis.

### BACKGROUND OF THE DISCLOSURE

In case of osseous defects or defective muscle or ligament structures, usually mobile knee joint endoprostheses of different degree of coupling for unlimited or limited mobility are implanted in accordance with the required stabilization of the knee joint. If a stabilization of the knee joint with knee joint endoprostheses which still allow a limited mobility is not possible anymore, for example, in case of a defective pulley or excessive bones defects, a surgical stiffening of the joint, or arthrodesis, respectively, may be effected by means of implants for providing a rigid connection of femur and tibia. Thus, mobility of the joint is totally inhibited.

A device for forming a knee arthrodesis is known from DE 199 62 324 A1.

Such a stiffening of the joint in case of an arthrodesis leads to a considerable restriction of mobility of the patient, who needs to newly train motion sequences of walking and who will possibly also perform compensatory motions for not stumbling when walking and also tries to compensate the limited mobility by a modified load on other elements of the musculoskeletal system. The more the arthrodesis deviates from the normal physiological state, the more will such compensatory motions occur, which in turn may lead to further discomfort and wear.

Hence, it is an object of the present disclosure to provide an endoprosthesis for a human knee for formation of an arthrodesis, which may well be adapted to the anatomic requirements of a patient.

If endoprostheses are implanted into patients, it can be necessary to provide endoprostheses or components thereof, respectively, having different dimensions for providing an endoprosthesis adapted to the individual anatomy of each patient. In a hospital wherein endoprostheses of a different type for different joints, for example, knee arthrodeses and artificial hip joints, are implanted, it is therefore necessary to provide a plurality of exemplars of different size for each type of endoprosthesis. Storage of the plurality of endoprostheses requires a large amount of space, and supply and sterile storage of the plurality of endoprostheses may lead to high costs.

It is, therefore, a further object of the present disclosure to provide a single system that unifies the supply of hip joint shafts and knee arthrodeses and thus allows to reduce the required storage space and costs.

### SUMMARY OF THE DISCLOSURE

According to one embodiment, the present disclosure relates to an endoprothetical connection element for forming a knee arthrodesis comprising a coupling element that comprises a first fastener and a second fastener. The first fastener is adapted for being connected with a fastening element corresponding to the first fastener that is provided on a shaft insertable into a femur of a patient. The second fastener is adapted for connection with a fastening element corresponding to the second fastener that is provided on a shaft insertable into a tibia of the patent. The coupling element is adapted such that a first longitudinal axis of the femoral shaft connected to the first fastener and a second longitudinal axis of the tibial shaft connected to the second fastener include an angle different from 180 degrees and lying in a coronal plane of the patient. Preferably, the coupling element is additionally implantable into the body of the patient in such a manner that the first longitudinal axis and the second longitudinal axis are also angled to each other in a sagittal plane of the patient.

According to a further embodiment, the present disclosure relates to a modular system for assembly of a knee arthrodesis and/or a hip joint endoprosthesis comprising a tibial shaft and a femoral shaft, as well as an endoprothetical connection element for forming a knee arthrodesis that comprises a coupling element comprising a first fastener and a second fastener. The first fastener is adapted for connection with a fastening element corresponding to the first fastener that is provided at a shaft insertable into a femur of a patient. The second fastener is adapted for connection with an fastening element corresponding to the second fastener that is provided at a shaft insertable into a tibia of the patient. The coupling element is adapted such that a first longitudinal axis of the femoral shaft connected to the first fastener and a second longitudinal axis of the tibial shaft connected to the second fastener include an angle different from 180 degrees, which, when implanted into the body of a patient, is lying in a coronal plane of the patient. Preferably, the coupling element is additionally implantable into the body of the patient in such a manner that the first longitudinal axis and the second longitudinal axis are also angled to each other in a sagittal plane of the patient.

The modular system further comprises a hip joint module comprising a joint ball that is attachable to the fastening element of the femoral shaft, wherein the fastening element of each of the shafts is suitably adapted both for the formation of the arthrodesis and for the formation of a hip joint endoprosthesis. A particularly well adaptation to the anatomical conditions is effected by a fine graduation of the shaft diameters of the shafts which are held in store. If different shafts were provided for the hip joint implant and the knee arthrodesis, a disproportionally high effort of supply would be required.

According to yet another embodiment, the present disclosure relates to a method of forming a knee arthrodesis that comprises providing a coupling element comprising a first fastener and a second fastener, wherein the first fastener and the second fastener are arranged at an angle to each other. A first shaft comprising a fastening element that is connectable to the first fastener is provided. Additionally, a second shaft is provided that comprises a further fastening element that is connectable to the second fastener. The first shaft is inserted into a femur of a patient. The second shaft is inserted into a tibia of the patient. The fastening element of the shaft insertable into the femur is connected to the first fastener for connection to the coupling element, wherein a longitudinal axis of the first shaft is aligned in a first direction. The other fastening element of the shaft insertable into the tibia is connected to the second fastener for attachment to the coupling element, wherein the longitudinal axis of the second shaft is aligned in a second direction. The first direction and the second direction are angled to each other in a coronal plane of the patient.

According to a further embodiment, the above-mentioned method further comprises providing a hip joint module comprising a joint ball adapted for attachment to the fastener of the femoral shaft.

### BRIEF DESCRIPTION OF THE DRAWINGS

Further embodiments of the present disclosure are specified in the appended claims. In the following description, further embodiments are described with reference to the attached figures, wherein:

FIG. 1 shows a schematic section of a skeletal structure in a front view, wherein various axial and angular alignments are shown;

FIG. 2A shows a schematic front view of a modular endoprosthesis for a human knee according to the present disclosure that comprises a first shaft suitable for insertion into a femur, a second shaft suitable for insertion into a tibia and a connection element;

FIG. 2B shows a schematic perspective side view of the modular endoprosthesis according to the present disclosure with both shafts and the connection element;

FIG. 2C shows a schematic cross-sectional view of a shaft of the endoprosthesis shown in FIGS. 2A and 2B,

FIG. 3A shows a schematic perspective side view of the endoprosthesis according to the present disclosure in a bent state;

FIG. 3B shows a side view and a cross section of the modular knee arthrodesis; and

FIG. 4 shows a schematic cross-sectional view of a hip joint module of a modular system according to the present disclosure.

### DETAILED DESCRIPTION OF THE PREFERRED EMBODIMENTS

While the present subject matter is described with reference to the embodiments as illustrated in the following detailed description and the drawings, it should be understood that the detailed description and the drawings are not intended to limit the present disclosure to the particular embodiments disclosed, but that the described embodiments merely exemplify various aspects of the present subject matter, the extent of which is defined by the appended claims.

According to an embodiment, an endoprothetical connection element for forming a knee arthrodesis comprises a coupling element that comprises a first fastener and a second fastener. The first and second fastener can be adapted for connection with corresponding fastening elements of shafts that can be inserted into bones of a patient, in particular into hollow bones. The connection element and the two shafts then form a knee arthrodesis.

One of the shafts that is connected to the first fastener can be inserted into the femur of the patient and the other shaft that is connected to the second fastener can be inserted into the tibia of the patient for implanting the knee arthrodesis into the patient. Thereby, the shafts may be selected from a choice of shafts in such a manner that a leg length adapted to the individual anatomy of the patient and a secure and stable fit of the knee arthrodesis are obtained.

Shafts of the type used in the knee arthrodesis according to this embodiment can also be used in other types of endoprostheses. For example, the shafts can also be combined with a hip joint module for forming an artificial hip joint. When used in an artificial hip joint, the shaft can be inserted into the femur of the patient. The hip joint module can comprise a joint ball that can be inserted into a joint socket attached to the pelvic bone of the patient. The shaft that is implanted into the patient can be selected from a choice of shafts of different dimensions for adapting the artificial hip joint to the individual anatomy of the patient. The fastening means of the shafts as well as those of the coupling element of the arthrodesis and the hip joint module are adapted to fit to each other, such that the same shafts can be used both for the hip joint prosthesis and for the arthrodesis.

Hence, a choice of shafts of different dimensions can be used both for forming knee arthrodeses and for forming artificial hip joints. This can allow a reduction of the number of components that need to be held in stock in an institution wherein endoprostheses are implanted, such as a hospital. Thus, the storage capacity required for storage of the components as well as the costs for obtaining the components can be reduced.

The first fastener and the second fastener can be adapted such that a longitudinal axis of a shaft connected to the first fastener is aligned in a first direction and a longitudinal axis of a shaft connected to the second fastener is aligned in a second direction.

The first direction and the second direction of the longitudinal axes of the shafts can be arranged in such a manner that they are angled to each other in a coronal plane of the patient after being implanted.

This allows an adjustment of the inclination or angle between the femur and the tibia in a coronal plane of the patient, respectively, in accordance with the anatomical requirements of the patient during surgery or, if necessary, during a revision performed at a later point of time. By means of the connection element, the femur and the tibia can be positioned relative to each other in a particularly simple manner for adjusting the axes of both shafts. Additionally, the connection element improves the stability of the implant with respect to mechanical load. Furthermore, a loosening of the implant can be avoided.

In an embodiment, the first direction and the second direction include an angle between 173 degrees and 176 degrees. In further embodiments, the angle between the first direction and the second direction preferably is 174 degrees and determines a valgus angle. Thus, particularly favorable values of inclination can be obtained that allow a correction of deformities or the like as well as a stable and permanent treatment adapted to the anatomy of the patient.

In an embodiment, the coupling element comprises at least one femur side portion and at least one tibia side portion. In a further embodiment, the at least one femur side portion and the at least one tibia side portion are engaged with each other. The division of the connection element in two portions allows a simple insertion of the shafts into the femur and the tibia, followed by a connection of the portions of the coupling element during surgery.

According to an embodiment, the at least one femur side portion of the coupling element comprises at least one rounded dorsal projection. The at least one tibia side portion of the coupling element can comprise at least one projection that is rounded and located a bit more ventral. In some embodiments, a dorsal projection of the at least one femur side portion and the ventral projection of the at least one tibia side portion are formed across the respective portion along the coronal plane. Thus, a stable and lasting connection between the two portions of the coupling element can be created. Furthermore, a large contact area can be formed between the two portions of the coupling element which ensures a good transmission of forces between the shaft inserted into the tibia and the shaft inserted into the femur. Optionally, the at least two portions of the coupling element are positive-fit connected to each other in a connected state. By means of the positive fit, an optimal transmission of moments is also achieved.

In some embodiments, the femur side portion of the coupling element comprises a step on a dorsal side thereof, which determines a maximal angle of flexion upon assembly during surgery. The step is adapted to simplify the connection of the femoral shaft and the tibial shaft by means of the portions of the coupling element upon insertion during surgery, such that a connection of the two portions of the connection element by screwing can be performed without an undue effort of adjustment.

In a further embodiment, the first direction and the second direction are angled to each other in a sagittal plane of the patient in the connected state. Thus, in a course of motions, the anatomical needs of the patient may be complied with as far as possible.

In a further embodiment, the first fastener and the second fastener comprise a thread, for example an internal thread. The fastening elements of the shafts can comprise a thread corresponding to the thread of the fastener, for example, an external thread. Thus, the fasteners and the fastening elements can be connected to each other by means of a screw connection. The internal threads can be provided inside of bores which are formed in the femur side portion and the tibia side portion of the coupling element, respectively. Advantageously, thus the shafts are fixable in an accommodation portion of the respective portion of the coupling element by means of a screw connection. For adjustment for position of the shafts, counter-nuts can further be provided at the threads of shafts. Thus, a stable connection between the individual components which counteracts a dislocation and, hence, counteracts undesirable attrition, can be created by means of a relatively low amount of work. Furthermore, the corresponding threads allow a fast and secure connection of the components under surgical conditions.

In a further embodiment, the coupling element comprises a femur side connector that comprises the first fastener and is fixed to the femur side portion of the coupling element, as well as a tibia side connector that comprises the second fastener and is fixed to the tibia side portion of the coupling element. In some embodiments, the angle between the first fastener and the femur side portion of the coupling element is adjustable in the coronal plane by means of the femur side connector. The tibia side connector can be fixed to the tibia side portion of the coupling element.

In case of a non-permanent attachment of the connector, the geometry of the connection element, and, hence, the geometry of the knee arthrodesis can be adapted to the anatomical requirements of the patient during surgery. Hence, it is not necessary to a provide a plurality of knee arthrodeses, from which one that suits to a human knee has be selected during surgery. The fixed connection of the tibia side connector to the tibia side portion of the coupling element allows a stable and permanent connection of both corresponding parts of the coupling element. A mobile design of the femur side connector has the advantage that the axes of both shafts can be positioned relative to each other for adjustment of the shafts in a particularly simple manner to obtain a desired inclination. Furthermore, due to the design of the femur side connector, predetermined angles of inclination can advantageously be set continuously or stepwise. Thus, the variability of the use of the modular stiffening element is increased and, furthermore, a particularly simple and safe handling for adjustment of the inclination is provided. Optionally, in some embodiments, at least two set screws are provided in the femur side portion of the coupling element for adjustment of the angle of the femur side connector.

In a further embodiment of the presentdisclosure, the first fastener can be pluggably connectable to the fastening element of the first shaft and the second fastener can be pluggably connectable to the fastening element of the second shaft.

In some embodiments, the shafts can be provided with grooves on an outer side thereof. Thus, a good contact to the bone material can be ensured. Thus an in-growth behavior of the shafts into the bone material is suitably supported and improved.

In a further embodiment, the first and the second fasteners of the knee arthrodesis module each comprise a bore hole. In further embodiments, the respective bore hole corresponds to tubular shafts for insertion of a screw into each of the shafts. Thus, an insertion of the shafts into the bones and a subsequent connection of the shafts to the bone material is particularly simple. Due to an interlocking of the bones with the corresponding shafts, a force absorbing positive connection is obtained. Additionally, this leads to a particularly secure long-term connection between the shafts and the knee arthrodesis module.

In one embodiment, the shafts are fixedly screwable to each other via the knee arthrodesis module after insertion. Thus, the desired or necessary, respectively, stiffening of the knee joint of the patient can be created and the anatomically aligned adjustment of the connected stiffening element can be fixed permanently.

A method for creating a knee arthrodesis according to an embodiment of the disclosure comprises providing a connection element or knee arthrodesis module that can comprise some or all of the above-described features of the connection element or knee arthrodesis module, respectively. Additionally, a first shaft and a second shaft are provided, which each can comprise some or all of the above-described features of shafts that are insertable into bones of a patient. The shafts comprise fastening elements that are connectable to corresponding fasteners of the connection element or knee arthrodesis module, respectively. The first shaft is inserted into the femur of the patient and the second shaft is inserted into the tibia of the patient. Subsequently, the fastening elements of the shafts are connected to the fasteners of the connection element. Thus, the shafts can be aligned relative to the connection element in the first and the second direction, respectively, such that an orientation of the femur and the tibia relative to each other that is adapted to the anatomy of the patient can be obtained. The first direction and the second direction are angled relative to each other in a coronal plane of the patient. Thus, a leg position of the patient having a slight valgus, corresponding to the natural shape of a stretched leg, can be obtained.

For selecting shafts that fit the patient, a plurality of trial shafts can be inserted both into the femur and into the tibia. It can be determined which of the trial shafts fits best to the individual anatomy of the patient. Subsequently, the final shafts can be selected from a choice of shafts having different length, shape and/or thickness, wherein the form of the selected shaft may essentially correspond to that of the best fitting trial shaft.

Before insertion of the shafts, soft tissue can be removed from the femur and the tibia, and the interior of the femur and the tibia can be rinsed, for example by means of a Jet-Lavage of a type known to persons skilled in the art. Thus, the bones of the patients can be prepared for insertion of the shafts.

In a method for providing a patient with an endoprosthesis according to an embodiment, a modular system for assembly of a knee arthrodesis and/or a hip joint endoprosthesis is provided. The system can comprise some or all of the features of the above described modular system. If a patient needs a knee arthrodesis, a knee arthrodesis is formed. For this purpose, the fastening elements of two of the shafts can be connected to the fasteners of the knee arthrodesis module. If, however, a patient needs a hip joint endoprosthesis, the fastening element of one of the shafts is connected to the fastener of the hip joint module for forming a hip joint endoprosthesis.

Combinations of individual and/or a plurality of the above-mentioned embodiments or designs of the present disclosure are also part of the present disclosure.

In the following, further embodiments are described with reference to the figures.

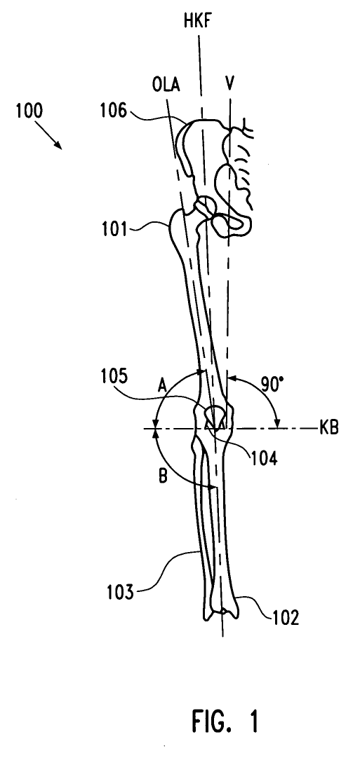

FIG. 1 shows a schematic skeletal composition of a right leg 100 of a human body comprising a thigh bone (femur) 101, a shin bone (tibia) 102 and a knee cap (patella) 105. Additionally, in FIG. 1 a fibula 103 and a pelvic bone 106 are shown. The femur 101 has two relatively wide, slightly convex joint pulleys, the condyles 104, at its end. Contrary thereto, an upper end of the tibia 102 comprises a tibial plateau, on which the condyles 104 rest. The great range of motion of up to 160° does not allow an osseous guide. Instead, stability in each position is secured by a complex system of muscles and ligaments (not shown). In case this stability is no longer present due to an accident or disease, and the muscles and ligament system or the bones are damaged to a large extent, a high stability and permanent connection can be obtained by means of a knee arthrodesis according to the present disclosure by stiffening of the knee.

In FIG. 1, a femoral axis OLA is tilted relative to a vertical axis V that includes an angle of 90° with the knee basis line KB, and also to an axis HKF, that connects the centers of the femoral head, the knee and the foot. The angle A resulting from the inclination of the longitudinal axis of the femur relative to the knee basis line KB is smaller than 90° and, in combination with the angle B between the knee basis line KB and the axis of the tibia 102, includes an angle less than 180° in the coronal plane. In general, the longitudinal axis of the femur is angled at an angle A of 81° in a physiological valgus position relative to the knee basis line KB. Typically, the angle B has a value of approximately 94°.

Therefrom, it is apparent that in an upright position, normally the femur 101 and the tibia 102 are angled relative to each other in the coronal plane, such that the longitudinal axis of the femur includes an angle with the HKF axis that is denoted as valgus angle. A malposition of the leg leads to an abnormal load and, hence, early attrition of the knee joint. Hence, each excessive abnormality of the axes forms a possible pre-stage of secondary complications.

The fastening elements of the shafts, and, hence, at least approximately, also the shafts themselves as well as the bones into which the shafts are inserted, can include an angle between approximately 173° and approximately 176°, for example an angle of approximately 174°. Expressed in a different manner, the longitudinal axis of the femur and the HKF axis include an angle of preferably approximately 6°. Thus, a value of inclination is obtained that allows a stable and permanent treatment that is adapted to the anatomy of the patient.

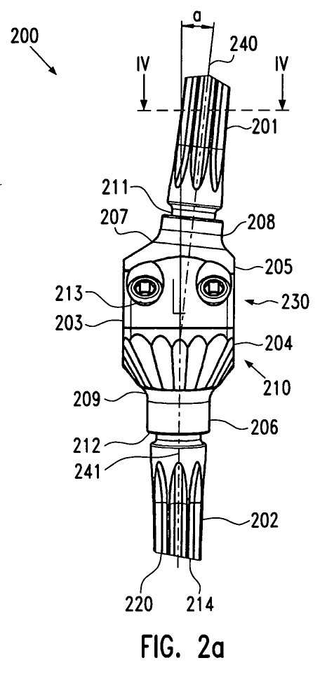

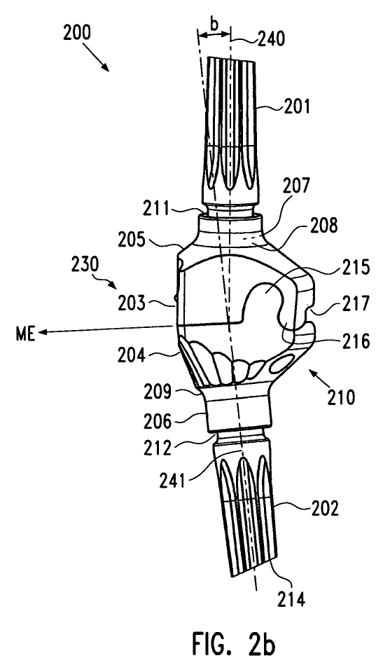

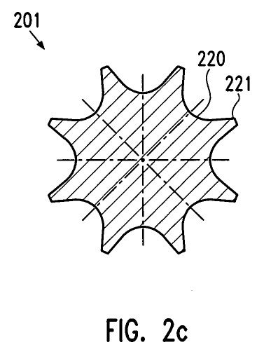

FIGS. 2a and 2b show a knee arthrodesis 200 for a human knee according to an embodiment. The knee arthrodesis 200 comprises a first shaft 201 that is attachable to a femur and a second shaft 202 that is attachable to a tibia. Furthermore, the knee arthrodesis 200 comprises a connection element 210 for connecting the first shaft 201 to the second shaft 202. The shafts 201, 202 have an exterior provided with grooves 220 and ribs 221 provided between the ribs 220, which allows to obtain a good contact with bone material. The grooves 220 and the ribs 221 can run along a longitudinal direction of the shafts 201, 202.

FIG. 2c shows a schematic cross-sectional view of the first shaft 201 along the line IV-IV that is drawn in FIG. 2. Due to the grooves 220 and the ribs 221, the cross-section of the first shaft 201 can have a star-like shape. The grooves 220 and the ribs 221 can be arranged symmetrically in a circumferential direction of the shaft 201. This may help to secure the first shaft 201 with respect to a rotation relative to the femur. In addition to a stable fixation in the bone, the ribs 220 ensure a sufficient metabolism in the supplying bone structures.

The first shaft 201 can preferably have a conical shape, wherein a diameter of the first shaft 201 at an end facing the connection element 210 can be greater than a diameter of the first shaft at an end distal of the connection element 210. Due to the conical shape, the first shaft 201 can be securely anchored in the femur, wherein the conical geometry of the shaft can counteract both a rotation of the first shaft 201 as well as a sinking of the first shaft 201 into the femur.

A surface of the exterior 214 of the first shaft can be rough. For this purpose, in some embodiments, the surface of the exterior 214 can be treated by rough blasting, for example with high quality corundum. A rough surface of the exterior 214 can enhance an engraftment of the bone of the patient to the first shaft 201 in case of cement-free implantation of the first shaft 201 into the femur, which allows to obtain a stable and resistant connection between the knee arthrodesis 200 and the patient.

The configuration of the second shaft 202 can essentially correspond to that of the first shaft 201, wherein the dimensions of the second shaft 202 can be different from those of the first shaft 201.

In some embodiments, the first shaft 201 and/or the second shaft 202 can have a curvature. Thus, the shafts 201, 202 can be particularly well adapted to the human anatomy. In one embodiment, the first shaft 201 is curved and the second shaft 202 is essentially straight. The first shaft 201 can have a length of approximately 140 mm to 300 mm, for example 200 mm, and the second shaft 202 can have a length of approximately 140 mm. In other embodiments, both shafts 201, 202 can be curved or straight, or the first shaft 201 can be straight and the second shaft 202 can be curved. In further embodiments, the shafts 201, 202 can have different dimensions than those mentioned above.

The connection element 210 comprises a coupling element 230. The coupling element 230 comprises at least one femur side portion 203 and at least one tibia side portion 204, which are engaged with each other. The connection of the coupling element 230 into two parts allows a simple insertion of the first shaft 201 into the femur and the second shaft 202 into the tibia as well an easy connection of the portions of the coupling element 230 during surgery.

Between the femur side portion 203 and the first shaft 201 as well as between the tibia side portion 204 and the second shaft 202, a connector 205, 206 can respectively be formed, which is preferably integral with the respective part of the coupling element. The connector 206 has a cylindrical shape, but can also have another convenient shape. The connector 205 has at least partially a cylindrical configuration. In case of a non-integral configuration, the connection by means of an connector allows a safe adjustment of the inclination and the angle in the coronal plane, respectively, during surgery. Advantageously, thus a maximum stability of the knee arthrodesis 200 with respect to mechanical load is ensured. The tibia side connector 206 can be fixedly connected to the tibia side portion 204 of the coupling element 230, or can be formed integrally with the coupling element 230. The angle a in FIG. 2a is formed by sides of the femur side connector 205, wherein preferably the medial side 207 of the femur side connector has an asymmetrical curved shape, and the lateral side 208 of the femoral connector has a symmetrical curved shape. Thus, the preset angle a is formed by the different lateral lengths of the femur side connector 205, such that the first shaft 201 and the second shaft 202 are angled relative to each other in the coronal plane. A connection of both parts of the coupling element is provided by two screws 213 in the femur side portion 203 of the coupling element.

The first shaft 201 and the second shaft 202 can be screwed to the respective portion 203, 204 of the coupling element via the connectors 205 and 206. For this purpose, the shafts 201, 202 can be provided with an external thread. Correspondingly, in this case, the connectors 205, 206 each comprise a bore 211 and 212, which comprises an internal thread corresponding to the external thread of the first shaft 201 and the second shaft 202. Additionally, counter-nuts can be provided on the threads of the shafts.

The internal thread in the connector 205 forms a first fastener. The external thread of the first shaft 201 forms a first fastening element that can be connected to the first fastener. The arrangement of the internal thread in the connector 205 defines a first direction 240. If the external thread of the first shaft 201 is screwed to the internal thread of the connector 205, the external thread is aligned in the first direction 240. Thereby, the first shaft 201 is also aligned in the first direction 240.

Correspondingly, the internal thread in the connector 206 forms a second fastener and the external thread of the second shaft 202 forms a second fastening element that is connectable to the second fastener. The arrangement of the internal thread in the connector 206 defines a second direction 241. If the external thread of the second shaft 202 is screwed to the internal thread of the connector 206, the external thread and, hence, also the second shaft 202, is aligned in the second direction 241.

In an implanted state, the knee arthrodesis 200 can be arranged such that the plane of drawing of FIG. 2a corresponds to the coronal plane of the patient. The first direction 240 and the second direction 241 can be angled relative to each other in the coronal plane, wherein the first direction 240 and the second direction include the angle a shown in FIG. 2a. In one embodiment, the angle a can have a value of approximately 6° wherein the angle a defines a valgus position corresponding to the natural anatomy of the human body. The connection element 210 can be provided in two configurations which are mirror-symmetrical relative to each other, wherein one of the configurations is adapted for implantation into a left leg and the other is adapted for implantation into a right leg. In some embodiments, the connection element 210 can have an adjustability for adjustment of the angle a. In other embodiments, the angle a can be predetermined.

The present subject matter is not limited to embodiments wherein the fasteners comprise internal threads and the fastening elements comprise external threads. In other embodiments, the fasteners can comprise external threads which are provided at the connectors 205, 206 and define the first and the second direction. In such embodiments, fastening elements can be provided at the shafts 201, 202 which are provided in the form of internal threads that correspond to the external threads in the connectors 205, 206.

The present subject matter is not limited to embodiments wherein the fasteners and the fastening elements comprise corresponding threads. In other embodiments, the first shaft 201 and the second shaft 202 can pluggably be connected to the femur side portion and the tibia side portion of the coupling element 230, respectively.

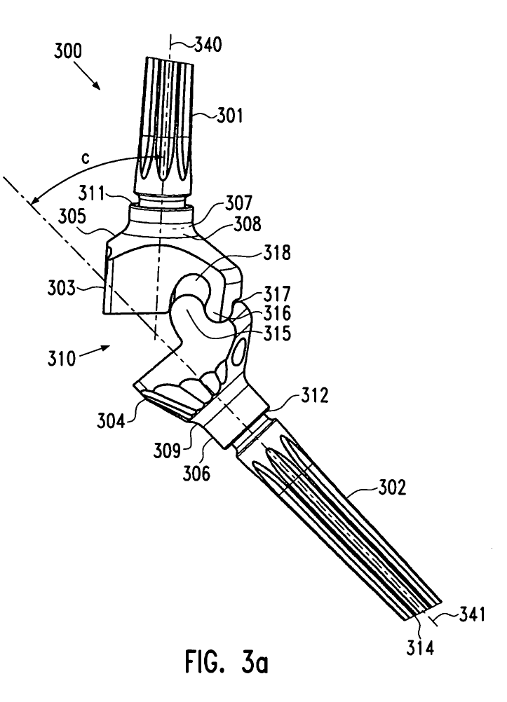

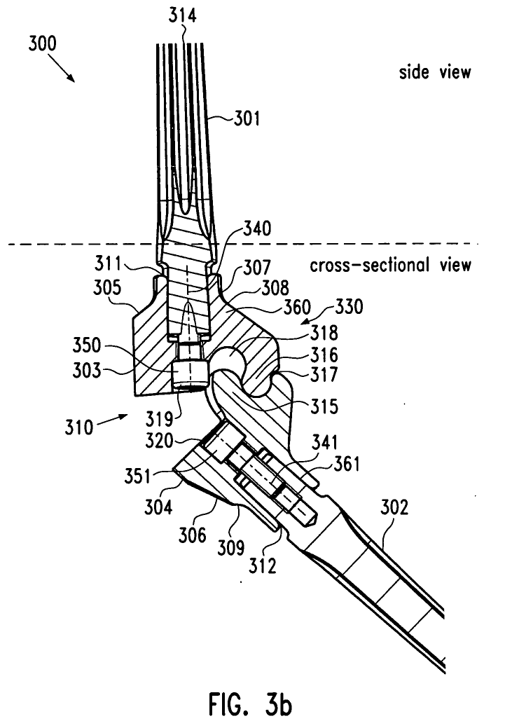

An embodiment wherein the fasteners and the fastening elements comprise conical connections will be described below with reference to FIGS. 3a and 3b.

FIG. 2b shows a schematic side view of a knee arthrodesis 200 comprising a first shaft 201, a second shaft 202 and a connection element 210. The connection element 210 comprises a coupling element 230. In an implanted state of the knee arthrodesis 200, the plane of drawing of FIG. 2b may approximately correspond to a sagittal plane of the patient. The coupling element 230 comprises a femur side portion 203 and a tibia side portion 204, which are connected to a first shaft 201 and a second shaft 202 by means of the respective connectors 205 and 206. The portions 203, 204 and the connectors 205, 206 are arranged such that the first direction 240 and the second direction 241 are angled to each other in a sagittal plane in a connected state at maximum extension ME as shown in FIG. 2b. The maximum angle of extension is denoted by reference numeral b. The femur side portion 203 of the coupling element 230 comprises a rounded dorsal projection 216. The tibia side portion 204 of the coupling element 230 comprises a rounded ventral projection 215, wherein the dorsal projection 216 and the ventral projection 215 are formed across the femur side portion 203 and the tibia side portions 204, respectively, along the frontal plane. Due to this configuration, the at least to one connection element portions 203 and 204 can be positive-fit connected to each other in a connected state. Preferably, both projections are formed at a slight angle to each other, for preventing a possible displacement in the horizontal direction. In a further embodiment, the femur side connection element portion 203 comprises a step 217.

FIG. 3a shows a knee arthrodesis 300 for a human knee in a bent state, which is characterized by an angle c. In the figure, a step 317 is shown again, which is formed at a femoral connection element portion 303 on the dorsal side. The step 317 acts as a guide and is capable of simplifying the connection of the first shaft 301 and the second shaft 302 via the portions 303, 304 of the coupling element 330 upon insertion during surgery, such that a screwing of the two parts 303, 304 can be performed without an exceeding effort of locking.

In general, the shafts 201, 202, 301, 302 are connected to the femur and the tibia, respectively, without cement. Thereby, the basic principle is ingrowth of bone material into the porous anchoring surface 214, 314 of the prosthesis, which may additionally be provided with grooves 220. Thus, a good contact to the bone material is ensured and, optionally, the ingrowth behavior of the stiffening element into the bone material is supported and improved thereby.

FIG. 3b shows a side view and a cross-section of a knee arthrodesis 300 for a human knee that comprises a first shaft 301 and a second shaft 302, as well as a connection element 310. The connection element 310 is formed by a coupling element 330 comprising at least one femur side portion 303 and at least one tibia side portion 304, wherein both portions 303 and 304 can respectively be connected to the respective shaft 301, 302 by means of corresponding connectors 305, 306.

As shown in the figure, the femur side portion 303 of the coupling element 330 and the tibia side portion 304 of the coupling element 330 each comprise a bore opening 319 and 320, wherein the respective bore opening 319, 320 corresponds to the first shaft 301 and the second shaft 302. The screws 350, 351 can each be screwed to a corresponding thread (not shown) in the first shaft 301 and in the second shaft 302.

The first shaft 301 comprises a male cone 311 at an end facing the connection element 310. Correspondingly, the second shaft 302 comprises a male cone 312 at an end facing the connection element 310. The male cones 311, 312 are each pluggable into a female cone 360, 361 in the portions 304, 305 of the coupling element 330. The female cone 360 in the portion 305 forms a first fastener and the male cone 311 forms a first fastening element that is connectable to the female cone 360. The female cone 361 in the part 304 defines a second fastener and the male cone 312 defines a second fastening element that can be connected to the female cone 361. Thereby, an axis of the female cone 360 defines a first direction 340 and an axis of the female cone 361 defines a second direction 341.

If the male cones 311, 312 are plugged into the female cones 360, 361, they are aligned along the first direction 340 and the second direction 341, respectively. Since the male cones 311, 312 are provided at the shafts 301, 302, the shafts 301, 302 are also aligned thereby. By means of the screws 350, 351, the shafts 301, 302 can be fixed in the aligned state.

Similar to the first direction 240 and the second direction 241 in the embodiments described above with reference to FIGS. 2a to 2c, in an implanted state of the knee arthrodesis, the first direction 340 and the second direction 341 can be aligned at an angle relative to each other in a coronal plane and/or a sagittal plane of the patient. The angles between the first direction 340 and the second direction 341 can correspond to those between the first direction 240 and the second direction 241.

After insertion, the shafts 301, 302 are fixedly screwable to each other by means of the connection element 330. Thus, the desired or necessary, respectively, stiffening can be provided to a human knee and the anatomically aligned adjustment of the connected endoprosthesis can be fixed.

High demands are made on the material properties of knee prostheses, since they should have a high resistance with respect to corrosion and wear. Furthermore, the prosthesis and possible abrasive particles must be well compatible to the human body. Due to the extreme, long-lasting load, to which the prosthesis is exposed in the body, particular demands are made, inter alia, to the long-term stability. Thus, a steel alloy on the basis of cobalt can be used for a cemented provision. Further alloy components can comprise chromium, molybdenum and nickel. Preferably, titanium alloys with aluminum-vanadium ingredients or niobium ingredients, respectively, are used for the manufacturing of prostheses.

A modular system for the assembly of a knee arthrodesis and/or a hip joint endoprosthesis according to an embodiment can comprise at least two shafts, which are implantable into at least one hollow bone of a patient, for example into a femur and a tibia of a patient. For example, the modular system can comprise the shafts 201, 202, 301, 302 described above with reference to FIGS. 2a to 3b. Additionally, further shafts can be provided.

In an embodiment, the modular system can respectively comprise one or more shafts having a length of approximately 140 mm, a length of approximately 200 mm, a length of approximately 260 mm and/or a length of approximately 320 mm. In some embodiments, the system can further comprise shafts of different thickness. In one specific embodiment, shafts of different thickness can be provided for each of the above mentioned lengths, wherein thicknesses of the shafts can be graded in units of 1 mm. The thicknesses of the shafts can be in a range from approximately 13 mm to approximately 22 mm (or, optionally, up to 30 mm). Additionally, straight and curved shafts can be provided.

The configuration of all shafts of the modular system can correspond to those of the above described shafts 201, 202, 301, 302. The fastening elements of all shafts can be essentially equal, such that each of the shafts can be connected to a fastener of a particular type.

The modular system can further comprise a knee arthrodesis module. In some embodiments, the knee arthrodesis module can be provided in the form of the connection element 210 (FIGS. 2a, 2b) and/or in the form of the connection element 310 (FIGS. 3a, 3b). Thus, the features of the knee arthrodesis module can correspond to those of the connection element 210 or the connection element 310, respectively. The fasteners of the knee arthrodesis module correspond to the fastening elements of the shafts, such that the fastening elements of the shafts can be connected to the knee arthrodesis module and can thereby be aligned in the first direction 240, 340 and in the second direction 241, 341, respectively.

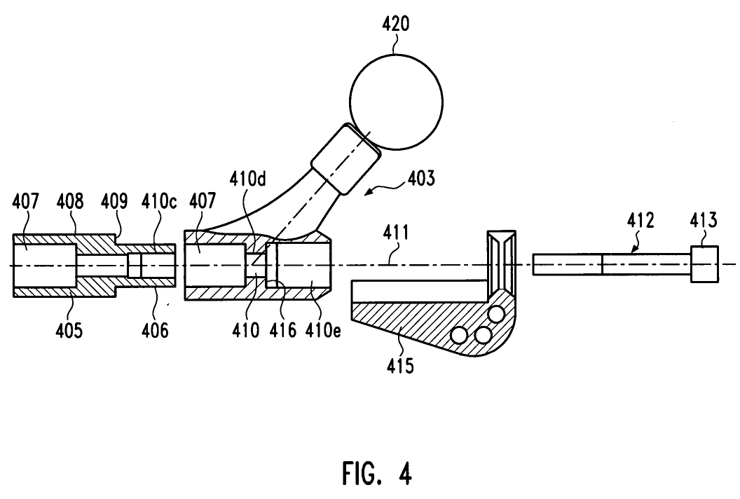

The modular system can further comprise a hip joint module. FIG. 4 shows a hip joint module 403, that may particularly be provided in combination with the connection element 310 shown in FIGS. 3a, 3b. The hip joint module 403 comprises an fastener provided in the form of a female cone 407. The shape of the female cone 407 can correspond to that of the female cones 360, 361 such that the male cones 311, 312 of the shafts 301, 302 are insertable into the female cone 407.

The hip joint module 403 can further comprise a longitudinal bore 410 having a longitudinal axis 411. A screw 412 can be inserted through the longitudinal bore 410 and screwed to a thread provided in the bores of the shafts. The hip joint module 403 can also comprise a thread 410d. Thus, one of the shafts can be attached to the hip joint module 403. A screw head of the screw 412 rests on a ring-shaped shoulder 416, that is located at an outermost end of a section 10d. A section 10e is adjacent to the ring-shaped shoulder 416.

The hip joint module 403 further comprises a joint ball 420 that is adapted for being inserted into a joint socket of a type known to persons skilled in the art.

The modular system can further comprise a bushing element 405. The bushing element 405 can comprise a female cone 407 and a cone section 406 that is tapered relative to a lateral area 408 under formation of a ring-shaped shoulder 9 in such a manner that the lateral area 408 has an essentially cylindrical shape if the cone section 406 of the bushing element 405 is inserted into the female cone 407 of the hip joint module 403.

The bushing element 405 can be inserted between the hip joint module 403 and one of the shafts 301, 302 for increasing a distance between the hip joint module 403 and the shaft 301, 302. Alternatively, the bushing element 405 can be inserted between the connection element 310 and one of the shafts 301, 302 for increasing the distance between the connection element and the respective shaft.

The modular system can further comprise a fin 415 that can be held in stock in different sizes. The fin 415 can be rotated around the longitudinal axis 411 for approximating a trochanter major removed in the course of the implantation of the hip joint module. The fin 415 can be attached to the hip joint section by means of a screw (not shown).

In accordance with requirements, a knee arthrodesis or a hip joint endoprosthesis can be assembled from the components of the modular system. For formation of a knee arthrodesis, two of the shafts 201, 202, 301, 302 can be connected to the knee arthrodesis module provided in form of the connection element 210, 310 as shown in FIGS. 2a to 3b. For formation of a hip joint endoprosthesis, one of the shafts 201, 202, 301, 302 can be connected to the hip joint module 403.

Hence, a choice of shafts of different dimensions needs to be held in stock only once for being able to form and implant a knee arthrodesis or hip joint endoprosthesis in accordance with requirements.

Prior to the implantation of the knee arthrodesis 200, 300, sufficiently long x-ray pictures of the affected knee region can be made. From the x-ray pictures, the size of the shafts 201, 202, 301, 302 to be inserted into the femur and the tibia can be measured by means of x-ray templates. Thus, both the expected diameter as well as the length and the type of shaft (straight or curved) can be determined preoperatively. In some embodiments, a shaft that ensures an osseous support that is as long as possible can be selected for obtaining a particularly stable attachment of the knee arthrodesis 200, 300 to the bones of the patient. The length of the shafts can be selected in accordance with the desired leg length of the patient.

In the implantation, access to the knee can be made by means of a median straight skin incision. In case of revision surgery, it can be of advantage to incorporate existing scars to obtain a better perfusion of the patient.

After a removal of parts of the femur and the tibia and/or a removal of an already existing endoprosthesis of the patient, the marrow spaces of the femur and the tibia can be prepared. For this purpose, soft tissue of any type can be removed. In case of revision surgery, granulation tissue and residues of cement which might possibly be present can be removed as completely as possible. In case of a post-infectious state, the marrow spaces can be drilled by means of marrow space drills adapted to the geometry. Additionally, a rinsing, for example by means of a Jet-Lavage, can be performed.

To validate the preoperatively planned shaft geometry, trial shafts can be inserted both into the femur and into the tibia. The trial shafts may have essentially the same size and shape as the shafts 201, 202, 301, 302 of the modular system, but may consist of a different material and can have a different surface structure, for example a smooth surface.

In doing so, one may start with a shaft having a considerably smaller diameter than the planned version. Successively, a trial shaft next in size can be used until the shaft diameter that optimally fits the individual anatomy of the patient is found. In doing so, both the planned depth of intrusion and a secure fit of the shaft 201, 202, 301, 302 in the osseous anchoring can be important criteria. Possibly, one may have to deviate from the planned length and to insert a longer implant.

The connection element 210, 310 can be attached to the trial shafts, and the leg length set by means of the trial shafts can be checked. Once the desired leg length has been obtained, the connection element 210, 310 can be detached again and the trial shafts can be exchanged for the final shafts 201, 202, 301, 302.

After inserting the shafts 201, 202, 301, 302, the parts 203, 204, 205, 206 of the coupling element 230, 330 of the connection element 210, 310 can be attached to the shafts 201, 202, 301, 302 and aligned with respect to rotation. Subsequently, the fastening elements of the shafts 201, 202, 301, 302 can be connected to the fasteners of the connection element 210, 310.

Subsequently, the portions 203, 204 of the coupling element 230, 330 can be fixed to each other. After a check of the rotation of the lower leg with respect to the upper leg, the shafts 201, 202, 301, 302 can be finally fixed to the connection element 210, 310. Subsequently, the components 203, 204 can be stably connected to each other by means of their positive fit and the screws 213.

Thereafter, the access to the knee of the patient can be closed and the skin of the patient can be sutured.

Further variants and modifications of the present subject matter will be obvious to persons skilled in the art in light of this description. Correspondingly, this description is to be construed as illustrative only and is for the purpose of teaching the persons skilled in the art the general way of performing the present subject matter. It is to be understood that the forms of the present subject matter shown and described herein are to be understood as the presently preferred embodiments.

What is claimed is:

1. Connection element for an endoprosthesis for forming a knee arthrodesis, comprising:
a coupling element comprising a first fastener and a second fastener;
wherein the first fastener is adapted for connection with a first fastening element corresponding to the first fastener that is provided on a shaft insertable into a femur of a patient;
wherein the second fastener is adapted for connection with a second fastening element corresponding to the second fastener that is provided on a shaft insertable into a tibia of a patient; and
wherein the coupling element is formed such that a first longitudinal axis of the femoral shaft attached to the first fastener includes with a second longitudinal axis of the tibial shaft connected to the second fastener an angle that is different from 180 degrees and is lying in a coronal plane of the patient when the endoprosthesis is implanted into the body of the patient.

2. Connection element according to claim 1, wherein the first longitudinal axis and the second longitudinal axis include an angle between approximately 173 degrees and approximately 176 degrees in the coronal plane.

3. Connection element according to claim 1, wherein the angle between the first longitudinal axis and the second longitudinal axis in the coronal plane is approximately 174 degrees.

4. Connection element according to claim 1, wherein the angle between the first longitudinal axis and the second longitudinal axis in the coronal plane defines a valgus angle.

5. Connection element according to claim 1, wherein in the coupling element comprises at least one femur side portion and at least one tibia side portion.

6. Connection element according to claim 5, wherein the at least one femur side portion and the at least one tibia side portion are engaged with each other.

7. Connection element according to claim 6, wherein the at least one femur side portion of the coupling element comprises at least one rounded dorsal projection.

8. Connection element according to claim 7, wherein the at least one tibia side portion of the coupling element comprises at least one rounded ventral projection.

9. Connection element according to claim 8, wherein the dorsal projection of the at least one femur side portion and the ventral projection of the at least one tibia side portion are formed across the femur side portion and the tibia side portion, respectively, along the coronal plane.

10. Connection element according to claim 5, wherein the at least one femur side portion of the coupling element and the at least one tibia side portion of the coupling element are positive-fit connected in a connected state.

11. Connection element according to claim 5, wherein the femur side portion of the coupling element comprises a step on a dorsal side that serves as a guide when both parts are connected during surgery.

12. Connection element according to claim 1, wherein the first longitudinal axis and the second longitudinal axis are provided at an angle to each other in a sagittal plane of the patient.

13. Connection element according to claim 1, wherein the first fastener and the second fastener comprise a thread.

14. Connection element according to claim 5, wherein the coupling element comprises a femur side connector that comprising the first fastener and is attached to the femur side portion of the coupling element, as well as a tibia side connector that comprises the second fastener and is attached to the tibia side portion of the coupling element.

15. Connection element according to claim 14, wherein a angle between the first longitudinal axis and the femur side portion of the coupling element is adjustable in the coronal plane by means of the femur site connector.

16. Connection element according to claim 14, wherein the tibia site connector is permanently connected to the tibia side portion of the coupling element.

17. Connection element according to claim 15, wherein at least two set screws for adjustment of the angle between the first longitudinal axis and the femur side portion of the coupling element are provided in the femur side portion of the coupling element.

18. Connection element according to claim 14, wherein at least one side of the femur side connector has a curved shape.

19. Connection element according to claim 18, wherein the medial side of the femur side connector has an asymmetrically curved shape.

20. Connection element according to claim 19, wherein the lateral side of the femur side connector has a symmetrically curved shape.

21. Connection element according to claim 1, wherein the first fastener is pluggably connectable to the first fastening element and the second fastener is pluggably connectable to the second fastening element.

22. Modular system for assembly of a knee arthrodesis and/or a hip joint endoprosthesis, comprising:
at least two shafts that are respectively insertable into a hollow bone of a patient and comprise a fastening element of essentially the same type at an end;
a knee arthrodesis module comprising a first fastener and a second fastener,
wherein the first fastener is adapted to provide a connection between the knee arthrodesis module and the fastening element and to align the fastening element in a first direction for attaching a first of the at least two shafts to the knee arthrodesis module;
wherein the second fastener is adapted to provide a connection between the knee arthrodesis module and the attachment module and to align the fastening element in a second direction for attachment of a second of the at least two shafts to the knee arthrodesis module,
wherein the knee arthrodesis module along with the first of the at least two shafts and the second of the at least two shafts are implantable into the body of a patient in such a manner that the first and the second direction are angled to each other in a coronal plane of a patient; and
wherein the modular system further comprises a hip joint module for attachment of a joint ball, wherein the hip joint module comprises a third fastener that is adapted to form a connection between the hip joint module and the fastening element for attachment of the first or the second of the at least two shafts to the hip joint module.

23. Modular system according to claim 22, wherein an exterior of each of the at least two shafts comprises grooves.

24. Modular system according to claim 22, wherein the first fastener and the second fastener of the connection element each comprise a bore hole.

25. Modular system according to claim 22, wherein the first of the at least two shafts and the second of the at least two shafts are permanently connectable to each other by screwing via the knee arthrodesis module after insertion.

26. A method of creating a knee arthrodesis, comprising:
providing a coupling element that comprises a first fastener and a second fastener, wherein the first fastener and the second fastener are provided at an angle relative to each other;
providing a first shaft, wherein the first shaft comprises a first fastening element that is connectable to the first fastener, and a second shaft, wherein the second shaft comprises a second fastening element that is connectable to the second fastener;
inserting the first shaft into a femur of a patient;
inserting the second shaft into a tibia of the patient;
connecting the first fastening element to the first fastener for attaching the first shaft to the coupling element, wherein the first shaft is aligned in a first direction;
connecting the second fastening element to the second fastener for attaching the second shaft to the coupling element, wherein the second shaft is aligned in a second direction; and
wherein the first direction and the second direction are angled relative to each other in a coronal plane of the patient.

27. Method according to claim 26, further comprising inserting a plurality of trial shafts having a different diameter and/or a different length into the femur, selecting one of the trial shafts that best fits to the anatomy of the patient and selecting the first shaft from a plurality of shafts, wherein the first shaft has essentially the same thickness and essentially the same diameter as the best fitting trial shaft.

28. Method according to claim 27, further comprising inserting a plurality of trial shafts having a different diameter and/or a different length into the tibia, selecting one of the trial shafts that best fits to the anatomy of the patient and selecting the second shaft from a plurality of shafts, wherein the second shaft has essentially the same thickness and essentially the same diameter as the best fitting trial shaft.

29. Method according to claim 26, further comprising removing soft tissue from the femur and the tibia and the patient and rinsing the interior of the femur and the tibia.

30. Method of providing a patient with a endoprosthesis, comprising:
providing at least two shafts that are adapted for insertion into at least one hollow bone of the patient and comprise a fastening element of essentially the same type at an end thereof;
providing a knee arthrodesis module comprising a first fastener and a second fastener;
providing a hip joint module for attachment of a joint ball that comprises a third fastener;
wherein the first, second and third fastener are adapted to provide a connection to the fastening element and to align the fastening element in a pre-determined direction;
forming a knee arthrodesis by connecting the fastening element of the first of the at least two shafts with the first fastener of the knee arthrodesis module and connecting the fastening element of the second of the at least two shafts with the second fastener of the knee arthrodesis module, if the patient needs a knee arthrodesis and;
forming a hip joint endoprosthesis by connecting the fastening element of the first of the at least two shafts or the second of the at least two shafts with the fastener of the hip joint module if the patient needs a hip joint endoprosthesis.

31. Method according to claim 30, wherein the first fastener is adapted to align the fastening element in a first direction; and
wherein the second fastener is adapted to align the fastening element in a second direction that is arranged at an angle to the first direction;
wherein the method further comprises;
implanting the knee arthrodesis into a patient wherein in the first of the at least two shafts is inserted into a femur of the patient and the second of the at least two shafts is inserted into a tibia of the patient, wherein the first direction and the second direction are angled to each other in a coronal plane of the patient.
